(19) United States
(12) Patent Application Publication
(10) Pub. No.: US 2004/0106810 A1
(43) Pub. Date: Jun. 3, 2004

(54) Process for the preparation of protected 1-(1-aminoakyl)-oxiranes

(76) Inventors: Giorgio Bertolini, Milano (IT); Pietro Bellani, Milano (IT)

Correspondence Address: CLARIANT CORPORATION, INTELLECTUAL PROPERTY DEPARTMENT, 4000 MONROE ROAD, CHARLOTTE, NC, 28205, US

(21) Appl. No.: 10/416,420

(22) PCT Filed: Sep. 27, 2001
(86) PCT No.: PCT/IB01/01789

(30) Foreign Application Priority Data
Nov. 16, 2000 (IT) MI2000A002466

(51) Int. Cl. C07D 303/00

(52) U.S. Cl. 549/513

(57) ABSTRACT

A method is described for the preparation of a compound of formula (formula see on enclosed paper version) where R is a protective group of the amino groups of the amino acids; R′ is a C1-C10 alkyl radical, an aryl, an aralkyl or a group ArX(CH2)m; where Ar is an aryl, X=O, S, NR″; R″ is a C1-C5 alkyl or aryl radical and m is an integer between 0 and 5; in which (a) the carboxyl group of a compound of formula (formula see on enclosed paper version) is activated by treatment with a group that is an activator of carboxyl groups; (b) the compound thus activated is made to condense with a trimethylsulphoxonium or tri-methylsulphonium ylide; (c) the keto-ylide thus obtained is reduced by reaction with a ketone-reducing agent; (d) the compound thus obtained is then cyclized to give the compound of formula IV by reaction with a base.

The present invention relates to a method of preparation of protected 1-(1-aminoalkyl) oxiranes having the formula

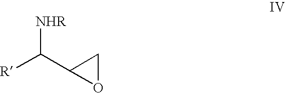

where R is one of the usual protective groups of the amino groups of the amino acids, such as tert-butoxycarbonyl (BOC), benzyloxycarbonyl (Cbz), methoxycarbonyl (Moc); R′ is a linear or branched C₁-C₁₀ alkyl radical, an aryl (substituted if necessary), an aralkyl (substituted if necessary) or a group ArX(CH₂)ₘ where Ar is an aryl (substituted if necessary), X=O, S, NR″; R″ is a C₁-C₅ alkyl or aryl radical and m is an integer between 0 and 5.

### STATE OF THE ART

The oxirane derivatives of formula IV and, in particular, the compounds of formula

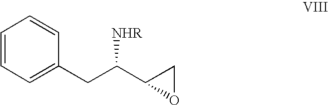

where R has the meaning stated above, are intermediates that can be used in the preparation of aspartate protease inhibitors, such as those described in the following patent documents: WO 96/16980, EP-337714, EP-356223, EP434365, U.S. Pat. No. 5,886,046, U.S. Pat. No. 6,034,247, EP-480711, EP482797, EP480624, U.S. Pat. No. 5,770,732, EP-528661, the contents of which must be regarded as an integral part of the present description.

Protected phenylalanine and lysine derivatives are disclosed in J. Enzyme Inhib., vol. 6, no. 3, 1992, pages 181-194, XP-0011037438, in J. Biol. Ch m., vol. 263, no. 6, 1968, pages 2768-2772, XP-001034096 and in Biochem. Journal, vol. 250, no. 3, 1988, pages 871-876, XP-001034063.

The methods known in the art for the preparation of th aforesaid compounds of formula IV are of relatively little interest from an industrial standpoint, as they generally exhibit low yields, of the order of 15-20% (U.S. Pat. No. 5,693,847; EP-206090), and/or make use of unstable intermediates, such as th α-amino aldehydes, which are easily racemized, thus necessitating subsequent stages of separation and purification (U.S. Pat. No. 5,693,847; WO 98/46577; WO 96/04277; U.S. Pat. No. 5,643,878; EP-626178; EP-528661; EP-434365; EP56223).

### DESCRIPTION OF THE INVENTION

A new method has now been found for synthesis of the intermediates of formula IV which does not have the drawbacks described above; this method, which constitutes the object of the present invention, is presented schematically below:

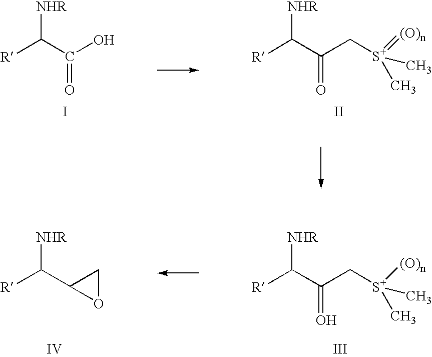

The process envisages firstly the activation of the carboxyl group of an amino-protected α-amino acid of formula I (methods for protecting the amino groups of the amino acids are well known in the prior art and are described for example in Greene et al., Protective Groups in Organic Synthesis, 1999, J. Wiley & Sons, incorporated here for reference; in practice they consist of substitution of an amino hydrogen with a protective group such as BOC, Cbz, Moc.); activation takes place by treatment of the compound of formula I with an activating agent that is specific for the carboxyl group, for example 1′1,1-carbonyldiimidazole, an alkylchloroformat, or an acyl halide such as the chloride or the fluoride. The solvent is generally an aprotic, apolar, organic solvent such as toluene or ethyl acetate, and/or a halogenated organic solvent such as methylene chloride or chloroform and/or an aprotic dipolar organic solvent such as THF; normally the reaction is carried out at a temperature between −50° C. and the solvent's reflux temperature, preferably between −15 and +30° C.

The protected amino acid, thus activated, is then made to condense with a trimethylsulphoxonium or trimethylsulphonium ylide. The preferred ylides are those of trimethylsulphoxonium or trimethylsulphonium chloride and iodide; these ylides should not however be regarded as limiting but merely as examples; other ylides can in fact be used for the purposes of the invention, for example those of acetates, perchlorates, bromides, arylsulphonates, etc.

In their turn, the aforementioned ylides are prepared by treatment of the corresponding sulphonium or sulphoxonium salt with strong bases such as sodium hydride or alcoholates of alkali metals in dipolar aprotic solvents such as dimethylsulphoxide, dimethylformamide, THF or with inorganic bases such as sodium hydroxide or potassium hydroxide in phase transfer conditions in apolar solvents such as toluene or in halogenated solvents such as methylene chloride at temperatures between 0° C. and the reflux temperature of the solvent, as described for example in E. J. Corey et al., J. Am. Chem. Soc., (1965) 87, 1353-1364, incorporated here for reference.

The reaction between the protected compound of formula I and the trimethylsulphoxonium or trimethylsulphonium ylide is normally carried out at a temperature between −50 and +60° C., generally at a temperature between −15 and +30° C.; the reaction solvent is normally a mixture consisting of the solvent in which activation of the protected α-amino acid was carried out and that in which the ylide was prepared. We thus obtain the keto-ylide of formula II, where n is an integer between 0 and 1, which is isolated using techniques that are known to a person skilled in the art and can be used for the subsequent reactions without further purification.

The keto-ylide of formula II is then reduced to the corresponding alcohol using ketone-reducing agents that are known to a person skilled in the art, for example hydrides (sodium boron hydride) or mixtures of these hydrides in the presence of activating agents such as formic acid, acetic acid, ammonium chloride; this reduction is normally carried out in alcoholic solvents such as methanol; in dipolar aprotic solvents, such as acetonitrile and THF; in chlorinated solvents, such as methylene chloride. The reaction temperature is generally between −78° C. and the reflux temperature of the solvent, preferably between −15 and +30° C. We thus obtain the compound of formula III, where n has th meaning stated above, which can be isolated by techniques known in the art and can then be used for th subsequent reactions without further purification; alternatively, the reaction mixture resulting from the reduction reaction containing the compound of formula II can be used directly for the next reaction without isolating the compound of formula III.

Compound III thus obtained is then cyclized to give the epoxide of formula IV by using a base (hydroxides of alkali metals or of ammonium, carbonates of alkali metals, alcoholates of sodium or potassium) in solvents such as acetonitrile, water, alcohols, chlorinated solvents, toluene or their mixtures at a temperature between −50° C. and the reflux temperature of the solvent (or of the mixture of solvents), preferably between −20 and +30° C., if necessary in the presence of a phase transfer catalyst, for example salts of ammonium, phosphonium or crown ethers.

In its preferred embodiment, the method according to the present invention is used for preparing protected 1-(1-aminoalkyl)oxiranes according to the reaction scheme shown below:

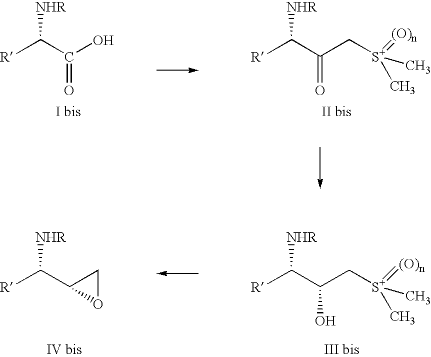

and, more particularly, it can be used for the synthesis of a protected 1-(1-aminoalkyl)-oxirane of formula

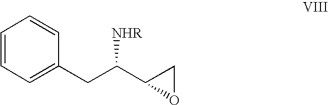

where R has the meaning described above, preferably BOC, Cbz or Moc.

In the optimum embodiment of the said method, a compound of formula

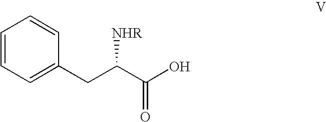

where R has the meaning described above, is activated with 1,1-carbonyldiimidazole (CDI) or with an alkylchloroformate, usually in THF, and is then reacted with a trimethylsulphoxonium or trimethylsulphonium ylide; since these ylides are preferably prepared in dimethylsulphoxide, the reaction is carried out by dropwise addition of the THF solution containing the activated α-amino acid to the dimethylsulphoxide solution containing the ylide.

We thus obtain a compound of formula

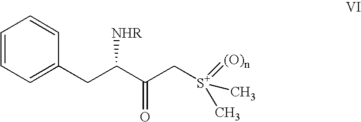

where n has the meaning described above, which is then reacted with sodium boron hydride, generally in acetonitrile and in the presence of formic acid, to give the corresponding reduction compound of formula

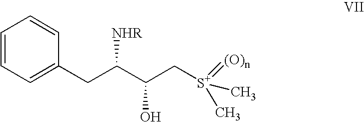

where n has the meaning described above.

The compound of formula VII is then cyclized to give the compound of formula VII by reaction with a base, generally NaOH or KOH, preferably working in a chlorinated organic solvent, such as methylene chloride, or in apolar organic solvents, such as toluene, and in the presence of a phase transfer catalyst, for example tetradecyttrimethylammonium bromide.

In the case when n is equal to 1, the compounds of formula II, II bis, III and III bis are novel and constitute a further object of the Invention.

As can be appreciated from the examples given below, which are purely for illustration and are non-limiting, the method according to the present invention makes it possible to obtain the desired compound at yields that are far higher than in the methods known in the prior art and without the use of unstable compounds such as precisely the x-amino aldehydes.

### EXAMPLE 1

Dimethylsulphoxonium[2-oxo-3-[(1,1-dimethylethoxycarbonyl)amino]-4-phenyl]butyl ylide

BOC-phenylalanine (30 g) and carbonyldiimidazole (21.8 g) are dissolved in tetrahydrofuran (420 ml) and the solution thus obtained is heated under reflux for 1.5 h, and is then cooled to 20° C. In another flask, sodium hydride (5.4 g) is added slowly at room temperature to dimethylsulphoxide (296 ml) followed by portion-wise addition of trimethylsulphoxonium hydride (29.8 g). The solution of activated BOC-phenylalanine is slowly added dropwise at room temperature to the solution of sulphoxonium methyl ylide previously prepared. At the end of addition, the reaction mixture is stirred for about two hours, then poured into water (800 ml) and extracted with ethyl acetate (3×300 ml). The organic phases are combined then washed with water (3×150 ml) and evaporated. The solid thus obtained is dried under vacuum to give 37.3 g of dimethylsulphoxonium[2-oxo-3-[(1,1-dimethylethoxycarbonyl)amino]+phenyl]butyl ylide (yield 97%) which is used for the next reaction without further purification.

### EXAMPLE 2

Dimethylsulphoxonium[2-hydroxy-3-[(1.1-dimethylethoxycarbonyl)amino-4-phenyl]butyl ylide

Sodium boron hydride (4.4 g) is added in portions at 25° C. to a solution of dimethyl sulphoxonium[2-oxo-[(11,1-dimethylethoxycarbonyl)amino]-4-phenyl]butyl ylide (12.7 g) in acetonitrile (76 ml) and formic acid (7.6 ml). The reaction mixture is stirred for about one hour; the suspension is filtered on Celite and is washed with acetonitrile (10 ml). The solution containing the reduction product, dimethylsulphoxonium[2-hydroxy-3-[(1,1-dimethylethoxycarbonyl)amino]4-phenyl]butyl ylide, is used directly for the next reaction without further purification.

### EXAMPLE 3

1-f 1-(1.1-Dimethylethoxycarbonyl)amino-2-phenylethyl]oxirane

The solution of dimethylsulphoxonium[2-hydroxy-3-[(1,1-dimethylethoxycarbonyl)-amino]4-phenyl]butyl ylide obtained from Example 2 is added dropwise to a mixture of crushed sodium hydroxide (12.2 g) and tetradecyltrimethylammonium bromide (0.7 g) in methylene chloride (150 ml), keeping the temperature above 40° C. The reaction mixture is stirred at 3040° C. for 30 minutes. The reaction mixture is then cooled to 25° C., filtered on silica gel (42 g) and washed with methylene chloride (70 ml). The solution is concentrated under vacuum and the solid thus obtained is suspended in methylene chloride (4.5 ml); the suspension is stirred at 45° C. for 15-30 minutes and is then cooled to 25° C. Hexane (45 ml) is added, the suspension is cooled to 0-3° C. and is stirred at this temperature for 30 minutes. The solid is filtered, washed with hexane and dried under vacuum, obtaining 5.4 g of 1-[1-(1,1 dimethylethoxycarbonyl)amino-2-phenylethyl]oxirane (yield in two passes 55%).

### EXAMPLE 4

1-[1-(1.1 Dimethylethoxycarbonyl)amino-2-phenylethyl]oxirane

The cyclization of the dimethylsulphoxonium[2-hydroxy-3[(1,1-dimethylethoxycarbonyl)amino]phenyl]butyl ylide in acetonitrile solution obtained from Example 2 can be carried out in conditions similar to those described in Example 3. Examples of reaction conditions and the corresponding yields are presented in Table 1.

TABLE 1

| | | | | Yi Id in |
|---|---|---|---|---|
| | | Phase transfer | Tempera- | solution in |
| Solv nt | Base | catalyst | ture | two passes |
| CH₃CN/CH₂Cl₂ | NaOH/water | C₁₄H₂₉Me₃NBr | 25° C. | 46.7 |
| CH₃CN/CH₂Cl₂ | NaOH | C₁₄H₂₉Me₃NBr | 25° C. | 41.0 |
| CH₃CN/CH₂Cl₂ | NaOH | C₁₄H₂₉Me₃NBr | 40-50° C. | 54.4 |
| CH₃CN/Toluene | NaOH | C₁₄H₂₉Me₃NBr | 25° C. | 40.6 |

### EXAMPLE 5

Dimethylsulphoxonium[2-oxo-3-[(benzyloxycarbonyl)amino]4-phenyl]butyl ylide

Cbz-phenylalanine (4 g) and carbonyldiimidazole (2.6 g) are dissolved in tetrahydrofuran (50 ml) and the solution thus obtained is heated under reflux for 1.5 h, and is then cooled to 20° C. In another flask, sodium hydride (0.6 g) is added slowly, at room temperature, to dimethylsulphoxide (19 ml) followed by addition of trimethylsulphoxonium hydride (3.0 g) in portions. The activated solution of Cbz-phenylalanine is slowly added dropwise at room temperature to the sulphoxonium methyl ylide solution previously prepared. At the end of addition, the reaction mixture is stirred for about two hours, then it is poured into water (80 ml) and extracted with ethyl acetate (3×30 ml).

The organic phases are combined, washed with water (3×1 5 ml) and evaporated. The solid thus obtained is dried under vacuum to give 3.9 g of dimethylsulphoxonium[2-oxo-3-[(benzyloxycarbonyl)amino]4-phenyl]butyl ylide (yield 78%) which is used for the next reaction without further purification.

### EXAMPLE 6

Dimethylsulphoxonium[2-hydroxy-3-[(benzyloxycarbonyl)amino]-4-phenyl]butyl ylide

Sodium boron hydride (0.15 g) is added in portions at 25° C. to a solution of dimethylsulphoxonium[2-oxo-3-[(benzyloxycarbonyl)amino]-4-phenyl]butyl ylide (1.0 g) in acetonitrile (10 ml) and formic acid (0.5 ml). The reaction mixture is stirred for about one hour, the suspension is filtered on Celite and is washed with acetonitrile (10 ml). The solution thus obtained is concentrated under vacuum and the residue is re-dissolved in isopropyl alcohol (10 ml). The solution containing the reduction product, i.e. dimethylsulphoxonium[2-hydroxy-3-[(1,1-dimethyloxycarbonyl)amino]A4-phenyl]-butyl ylide, is used directly for the next reaction without further purification.

### EXAMPLE 7

1-[1-(Benzyloxycarbonyl)amino-2-phenylethyl]oxirane

An aqueous solution of sodium hydroxide at 40% (1.3 g) is added to the solution of dimethylsulphoxonium[2-hydroxy-3-[(benzyloxycarbonyl)amino]4-phenyl]butyl ylide obtained from Example 5. The reaction mixture (two phases) is stirred at room temperature for 30 minutes. The aqueous phase is then separated and the organic phase is diluted with ethyl acetate (100 ml) and is washed with water (3×10 ml). The organic phase is made anhydrous over magnesium sulphate and is concentrated, under vacuum, obtaining 0.45 g of raw 1-[1-(benzyloxycarbonyl)amino-2-phenylethyl]-oxirane which can be crystallized by adding methylene chloride (0.2 ml) and hexane. The solid is filtered, washed with hexane (8 ml) and dried under vacuum obtaining 0.37 g of 1-[1-(benzyloxycarbonyl)amino-2-phenylethyl]oxirane (yield in two passes 37%).

### EXAMPLE 8

Dimethylsulphoxonium[2-oxo-3-[(1.1-dimethylethoxycarbonyl)amino]-4-phenyl]butyl ylide

Carbonyldiimidazole (13.4 g) is added in portions to a solution of BOC-phenylalanine (20 g) in tetrahydrofuran (40 ml) at 3° C. The solution thus obtained is stirred at 3° C. for 2 hours, then poured into a mixture of toluene (200 ml) and water (100 ml). The two phases are separated and the organic phase is washed with water (2×100 ml). In another flask, sodium hydride (2.7 g) is added slowly, at room temperature, to dimethylformamide (200 ml) then the trimethylsulphoxonium hydride (14.9 g) is added in portions and the mixture is stirred at room temperature for 2 hours until a clear solution is obtained. This solution is slowly added dropwise at 3° C. to the toluene solution of activated BOC-phenylalanine. At the end of addition, the reaction mixture is stirred for about two hours, and then poured into water (1000 ml). The mixture thus obtained is stirred at 3° C. for 15 minutes, then the solid that has formed is filtered and washed with cold water until the washings are neutral. The product thus obtained was dried under vacuum to give 16 g of dimethylsulphoxonium[2-oxo-3-[(1,1-dimethylethoxycarbonyl)-amino]-4-phenyl]butyl ylide (yield 70%) which was used for the next reaction without further purification.

### EXAMPLE 9

Dimethylsulphoxonium [2-hydroxy-3[(1,1-dimethylethoxycarbonyl)amino]-4-phenyl]butyl ylide

Sodium boron hydride (3.1 g) was added in portions at 3° C. to a solution of dimethylsulphoxonium [2-oxo-3-[(1,1-dimethylethoxycarbonyl)amino]-4-phenyl]butyl ylide (16 g) in acetonitrile (86 ml) and formic acid (8.5 ml) in about 45 minutes. The reaction mixture was stirred for about one hour, the suspension was filtered on Celite and then washed with acetonitrile (14 ml). The solution containing the reduction product, dimethylsulphoxonium[2-hydroxy-3-[(1,1-dimethylethoxycarbonyl)amino]-4-phenyl]butyl ylide, was used directly for the next reaction without further purification.

### EXAMPLE 10

1-[1-(1-1.1 Dimethylethoxycarbonyl)amino-2-phenylethyl]oxirane

The solution of dimethylsulphoxonium[2-hydroxy-3-[(1,1-dimethylethoxycarbonyl)-amino]4-phenyl]butyl ylide obtained from Example 9 was added dropwise to a mixture of crushed sodium hydroxide (10.2 g) and tetradecyltrimethylammonium bromide (0.8 g) in methylene chloride (170 ml), keeping the temperature above 40° C. The reaction mixture was stirred at 30-40° C. for 30 minutes. The reaction mixture was cooled to 25° C., the reaction mixture was filtered on silica gel (32 g) and then washed with methylene chloride (80 ml). The solution thus obtained was concentrated under vacuum. The solid thus obtained is absorbed in isopropanol (24 ml) and the solid that had formed is filtered off. The alcoholic solution is evaporated to dryness under vacuum and the raw product thus obtained is purified by column chromatography on silica gel (eluent: methylene chloride). The product thus purified is dissolved in acetone (11 ml) and is slowly added dropwise to water (44 ml) at 3° C. The solid is filtered, washed with water and dried under vacuum obtaining 1-[1-(1,1 dimethylethoxycarbonyl)amino-2-phenylethyl]oxirane.

1. A method of preparing a compound of formula 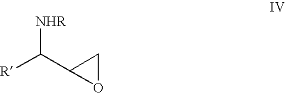
where R is a protective group of the amino groups of amino acids; R′ is a linear or branched C₁-C₁₀ alkyl radical, an aryl (substituted if necessary), an aralkyl (substituted if necessary) or a group ArX(CH₂)ₘ; where Ar is an aryl (substituted if necessary), X=O, S, NR″; R″ is a C₁-C₅ alkyl or aryl radical and m is an integer between 0 and 5; characterized in that:
a) the carboxyl group of a compound of formula 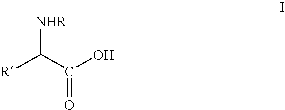
where R and R′ have the meanings indicated above, is activated by treatment with a group that is an activator of carboxyl groups;
b) the compound thus activated is made to condense with a trimethylsulphoxonium or trimethylsulphonium ylide;
c) the ketoylide thus obtained is reduced by reaction with a ketone-reducing agent;
d) the compound thus obtained is then cyclized to give the compound of formula IV by reaction with a base.

2. A method according to claim 1, characterized in that R′ is a benzyl group.

3. A method according to Claim 1, characterized in that R is a tert-butoxycarbonyl, benzyloxycarbonyl or methoxycarbonyl group.

4. A method according to claim 1, characterized in that th activation step (a) is carried out by treatment with 1,1-carbonyldiimidazole, with an alkylchloroformate or with an acyl halide, preferably chloride or fluoride.

5. A method according to claim 1, characterized in that the activation step (a) is carried out in an aprotic apolar organic solvent such as toluene or ethyl acetate, and/or in a halogenated organic solvent such as methylene chloride or chloroform and/or in a dipolar aprotic organic solvent such as THF.

6. A method according to claim 1, characterized in that the activation step (a) is carried out at a temperature between −50° C. and the reflux temperature of the solvent, preferably between −15 and +30° C.

7. A method according to claim 1, characterized in that the ylides used in the condensation step (b) are ylides of trimethylsulphoxonium chloride and/or hydride and/or ylides of trimethylsulphonium chloride and/or hydride.

8. A method according to claim 1, characterized in that the condensation step (b) is carried out at a temperature between 0 and +60° C., preferably at room temperature.

9. A method according to claim 1, characterized in that the said reduction step (c) is carried out in alcoholic solvents, such as methanol; in dipolar aprotic solvents, such as acetonitrile and THF; in chlorinated solvents, such as methylene chloride; or their mixtures.

10. A method according to claim 1, characterized in that the said reduction step (c) is carried out at a temperature between −78° C. and the reflux temperature of the solvent, preferably between −15 and +30° C.

11. A method according to claim 1, characterized in that the said ketone-reducing agent is selected from th hydrides, for example sodium boron hydride.

12. A method according to claim 11, characterized in that said hydrides are used in the presence of activating agents, such as formic acid, acetic acid and ammonium chloride.

13. A method according to claim 1, characterized in that the base used in the said cyclization step (d) is selected from hydroxides of alkali metals, alkaline-earth metals or of ammonium, carbonates of alkali metals or alkaline earth metals, alcoholates of sodium or potassium.

14. A method according to claim 1, characterized in that the said cyclization step (d) carried out in acetonitrile, water, alcohols, chlorinated solvents, toluene or mixtures thereof.

15. A method according to claim 1, characterized in that the said cyclization step (d) carried out at temperatures between −50° C. and the reflux temperature of the solvent or of the mixture of solvents, preferably between −20 and +30° C.

16. A method according to claim 1, characterized in that the said cyclization step (d) carried out in the presence of a phase transfer catalyst, for example salts of ammonium, phosphonium or crown ethers.

17. A method according to any one of the preceding claims for the preparation of a compound of formula 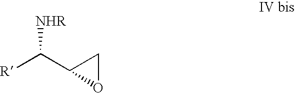
starting from a compound of formula 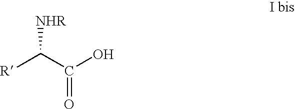
where R and R′ have the meanings given above.

18. A method for the preparation of aspartate protease inhibitors comprising a method according to claims 1-18.

19. A compound of formula 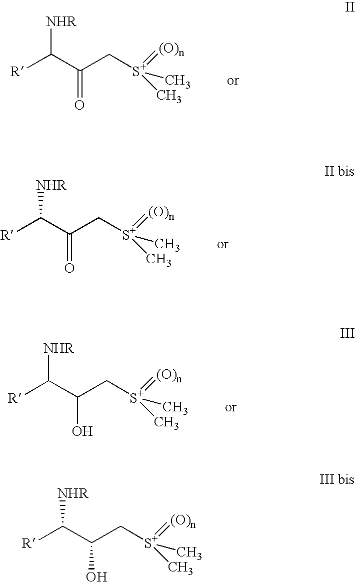
where R is a protective group of the amino groups of amino acids; R′ is a linear or branched C₁-C₁₀ alkyl radical, an aryl (substituted if necessary), an aralkyl (substituted if necessary) or a group ArX(CH₂)ₘ; where Ar is an aryl (substituted if necessary), X=O, S. NR″; R″ is a C₁-C₅ alkyl or aryl radical; m is an integer between 0 and 5 and n is 1.

20. A compound according to claim 19, characterized in that R′ is a benzyl group and R is a tert-butoxycarbonyl, benzyloxycarbonyl or methoxycarbonyl group.
